(19) United States
(12) Patent Application Publication
(10) Pub. No.: US 2024/0099310 A1
(43) Pub. Date: Mar. 28, 2024

(54) FORMULATION OF WATER-DISPERSIBLE GRANULES CONTAINING MICROBIOLOGICAL ACTIVES AND PREPARATION METHOD

(71) Applicant: Oxiteno S.A. Indústria e Comércio, Säo Paulo (BR)

(72) Inventors: Poliana Cardoso GUSTAVSON, Mauá (BR); Mariana MARTINS REIS ANDRADE, Mauá (BR); Fabiana IANHEZ BUZZUTTI, Mauá (BR); Flávia REGIANINI MONTIBELLER, Mauá (BR); Bruna MENDONÇA, Mauá (BR)

(73) Assignee: Oxiteno S.A. Indústria e Comércio, São Paulo, SP (BR)

(21) Appl. No.: 18/265,703

(22) PCT Filed: Nov. 10, 2021
(86) PCT No.: PCT/BR2021/050492

§ 371 Date: Jun. 7, 2023

(30) Foreign Application Priority Data
Dec. 7, 2020 (BR) BR 10 20200249754

(51) Int. Cl. A01N 63/38; A01N 25/04; A01N 25/14; A01P 3/00; A01P 5/00; A01P 7/04

(52) U.S. Cl. CPC A01N 63/38; A01N 25/04; A01N 25/14; A01P 3/00; A01P 5/00; A01P 7/04

(57) ABSTRACT

The present invention describes water-dispersible granule formulations containing at least one microbiological active, at least one oil, at least one anionic surfactant and at least one dispersed or water-soluble inert agent, as well as production method thereof. The formulations obtained by the described method have a longer shelf life, less contamination and allow for storage at room temperature.

### FIELD OF THE INVENTION

The present invention belongs to the field of agrochemical formulations and preparation methods thereof.

### BACKGROUND OF THE INVENTION

It is known to use microorganisms such as bacteria, fungi and viruses that act as pesticides or biostimulants, improving plant development and productivity. It is also known that, in the practical use of such living organisms, the use of suitable formulations is not free of problems. When formulated, most microorganisms lose their viability within a few days at room temperature, and the physicochemical characteristics of the formulation are generally less than expected. Therefore, it can be very difficult to produce formulations whose effects meet the minimum requirements for agricultural use.

In formulations containing fungi and/or bacteria, maintaining the viability of microorganisms at room temperature is, in fact, the biggest challenge for the success of the formulation. However, most of the literature on solid formulations shows satisfactory maintenance of viability only when such formulations are kept refrigerated (−4° C.) (see reviews in Schisler et al. (2004) Phytopathology 94: 1267-1271; Kiicuck & Kivanic (2005) African Journal of Biotechnology 4: 483-486), making logistics for stock and its use in agriculture very difficult.

Indeed, it is desirable the microbiological formulations preserve viability during shelf life under conditions in which refrigeration must be dispensed with.

By “microbiological formulations” it is meant compositions containing fungi, bacteria, viral particles, macromolecules or particles of different biological natures (proteins, polysaccharides, lipids and combinations thereof) produced in an extracellular medium by microorganisms, proteins, DNA, RNAi that act as pesticides. As an example, we can mention the main microorganisms currently formulated Amblyseius sp., Azozpirillum sp., Bacillus sp., Baculovirus sp., Beauveria sp., Cotesia sp., Cryptolaemus sp., Deladenus sp., Heterorhabditis sp., Isaria sp., Metarhizium sp., Neoseiulus sp., Orius sp., Paecilomyces sp., Pasteuria sp., Phytoseiulus sp., Pseudomonas sp., Stratiolaelaps sp., Telenomus sp., Trichoderma sp., Trichogramma sp., or mixtures thereof.

The most common solid formulations for use in microbiological formulations are usually of the wettable powder type, wherein the active is micronized as a powder and typically applied as suspended particles after dispersion in water. However, bacteria and fungi cannot be micronized, as shear can lead to Loss of cell viability. Thus, the granules have particles that vary in size from 0.2 to 4 mm, and can be formed by agglomeration, spray drying or extrusion techniques. The physicochemical implications of a wettable powder-type formulation, however, are quite limiting, which usually makes it enormously difficult or even restricts the satisfactory application in the field.

Some documents (U.S. Pat. No. 5,730,973A, EP0669079A1, W02012/162472A1, W02016/057203A1) contemplating solid formulations containing fungi or bacteria from solid or liquid fermentations, as well as academic literature (Chumthong et al. 2008. World Journal of Microbiology and Biotechnology 24: 2499-2507) report the preference for developing water-dispersible granule-type formulations over wettable powder to improve the physicochemical characteristics of the formulation and improve application. Water-dispersible granules-type solid formulations are characterized by quick disintegration and dispersion in water, and it have been increasingly requested for biological uses due to the better quality of the physicochemical parameters, the possibility of a higher concentration of microbiological actives and less limitation regarding the application in the field, as they usually have more adequate physicochemical characteristics, such as better wetting and suspensibility.

Water-dispersible granule-type formulations generally protect microorganisms from external interfering through the use of polysaccharides such as sucrose, glucose (U.S. Pat. No. 5,730,973A) or alginates (EP0669079A1), not only including the use of an encapsulation of the microorganism in oil prior to the addition of the other formulation components. As will be shown, the novelty of the method described herein is mainly in this stage of encapsulation in oil, which surprisingly increases the shelf life and allows for the storage of the formulation containing microorganisms at room temperature.

The previous encapsulation in oil additionally allows the formulation of more than one microorganism species or the formulation of microorganisms and chemical actives or biostimulant compounds in the same composition.

By “biostimulants” it is meant compositions containing plant activators and/or growth regulators, for example from the chemical group: dioxocyclohexanecarboxylic acid, indole alkanoic acid, aliphatic alcohol, quaternary ammonium, carbimide, carboxanilide, cycloalkene, cyclohexadione, cytokinin, dinitroaniline, inhibitor of ethylene, ethylene precursor, gibberellin, pyridazinedione, terpenoids, triazole, benzothiadiazole, or mixtures thereof. These compositions may also include signaling molecules such as nitric oxide, methyl salicylate, methyl jasmonate, proteins (and thus enzymes), polypeptides, lipopolysaccharides, polyamines, and complex compositions such as algae extracts, fulvic acid, humic acid, and microorganisms such as PGPR (plant growth promoting rhizobacteria), bacteria and fungi producing compounds that stimulate plant growth and reduce the deleterious effects of abiotic stresses such as water stress.

Two parameters directly affect the shelf life of water-dispersible granule-type formulations containing microorganisms: the water activity of the granules and the interaction with surfactants. The first can be mitigated by the microorganism encapsulation in a hydrophobic matrix, while the use of biocompatible surfactants is essential for maintaining the microorganism viability, that is, for the colony forming unit (CFU) count to be as close as possible to that performed immediately after fermentation.

By “biocompatibility” is meant the property of biological units (cells, tissues, organisms) to react to the presence of non-living materials. In other words, the beneficial or toxic influence of non-living compounds (including chemicals) on living beings. In this document, this concept is applied to assess the influence of formulations on the fungi or bacteria viability.

The CFU is a measurement unit used to estimate the number of viable bacteria or fungi, that is, capable of multiplying through binary fission under controlled conditions.

According to all the challenges described, there is still a need for simple preparation methods that are easy to scale and prepare, in addition to providing handling for biological control agents suitable for leave and soil application. Among other properties, formulations should ideally provide good physicochemical stability in the formulation concentrate, show adequate shelf life at room temperature, and ensure satisfactory distribution of the microorganism in both spray and soil applications. In addition, and maybe most essential, is the maintenance of viability in order to assure the biological effectiveness of the microbiological active.

In addition to the protection to the microorganism provided by the encapsulation in a hydrophobic matrix, the sequence of component addition during the preparation of the slurry to be obtained is fundamental for the good performance of a water-dispersible granule. This sequence is even more critical in formulations containing microbiological actives (bacteria, fungi), as the addition of surfactants or inert agents directly on the microorganisms can lead to a drastic decrease in its viability over the shelf life.

### DETAILED DESCRIPTION OF THE INVENTION

The present invention describes water-dispersible granule formulations containing microbiological actives and methods for preparing the formulations. Specifically, the invention refers to the microorganism encapsulation in oil to avoid its direct contact with water, fermentation remainder, air and other components of the formulation (inert agents, surfactants) that can reduce viability over shelf life. Then, inert agents and surfactants are added to facilitate application and improve physicochemical properties.

As an encapsulating agent, mineral and/or plant oils, fatty acids, esters, acetates or betaines can be used as single components or in mixtures, making up a product whose required HLB is close to that showed by the target microorganism.

Regarding inert agents, the use of polysaccharides such as chitosan, cellulose or starch, or minerals such as kaolin, clays or silica is recommended.

The present invention requires the use of biocompatible surfactants that have specific functions in the formulation to ensure its best application in the field: emulsifiers, dispersants, and moisturizing agents. These surfactants can be applied in the same sequence as independent components in the mixture or in compositions concerning the chemical characteristics described below.

Regarding (biocompatible) emulsifiers, those belonging to the groups of ethoxylated polyols, ethoxylated and propoxylated polyols, sorbitan esters, ethoxylated sorbitan esters, ethoxylated and propoxylated sorbitan esters, ethoxylated oils, ethoxylated alcohols, and mixtures thereof are recommended.

Dispersing agents should be used, preferably those from glycols, ethoxylated alcohols, ethoxylated phenols, ethoxylated polyols, ethoxylated and propoxylated polyols, sorbitan esters, ethoxylated sorbitan esters, ethoxylated and propoxylated sorbitan esters, and mixtures thereof.

As for moisturizing agents, acetates, ethoxylated alcohols and block copolymers are preferably used.

Other additional components (UV protectors, antifoaming), or a second microorganism or pesticide (e.g., herbicides, insecticides, nematicides or fungicides), or biostimulant compounds (terpenoids, proteins, lipopolysaccharides, polypeptides, polyamines or other signaling molecules) to be formulated in set can be added at the end of the formulation and before the extrusion and drying of the slurry obtained.

Subsequently, the invention is described as examples, which show solid formulations containing at least one microorganism, or in combination with other microorganisms, pesticides or biostimulants, as well as its method of preparation and its properties.

### Example 1—Solid Formulations (Water-Dispersible Granules) Containing at Least One Microbiological Active and Method of Preparation

A microbiological formulation according to the present invention contains microorganisms obtained from solid fermentation as a granule comprising:
(A) 0.2 to 45% weight, preferably 0.2 to 45% weight, of Trichoderma harzianium spores;
(B) 0.2 to 45% weight, preferably 0.8 to 45% weight, of mineral or plant oil or a mixture;
(C) 0.5 to q.s.p. of inert agents (starch, cellulose, clays, silica);
(D) 1 to 90% weight, preferably 10%, of surfactants consisting of emulsifiers, dispersants and moisturizing, added in this order or only in one composition;
(E) 0.1 to 10% weight, preferably 2%, UV protector. The microbiological formulation may contain microorganisms obtained from liquid fermentation as a granule comprising:
(A) 0.2 to 45% weight, preferably 0.2 to 45% dry weight, of liquid fermentation broth of Bacillus subtilis;
(B) 0.2 to 45% by weight, preferably 0.8 to 45% weight, of mineral or plant oil or a mixture;
(C) 0.5 to q.s.p. of inert agents (starch, cellulose, clays, silica);
(D) 1 to 90% weight, preferably 10%, of surfactants consisting of emulsifiers, dispersants and moisturizing agents, added in this order or only in one composition;
(E) 0.1 to 10% weight, preferably 2%, of UV protector.

The microbiological formulation may also contain more than one microbiological active obtained from solid and/or liquid fermentation, a pesticide (herbicides, insecticides, nematicides or fungicides) or biostimulant

Compounds (terpenoids, proteins, lipopolysaccharides, polypeptides, polyamines or other signaling molecules) as a granule comprising:
(A) 0.2 to 45% weight, preferably 0.2 to 45% dry weight, fungal or bacterial spores, or fungus or bacteria liquid fermentation broth;
(B) 0.2 to 45% weight, preferably 0.8 to 45% weight, of mineral or plant oil or a mixture;
(C) 0.5 to q.s.p. of inert agents (starch, cellulose, clays, silica);
(D) 1 to 90% weight, preferably 10%, of surfactants comprised of emulsifiers, dispersants and moisturizing agents, added in this order or only in one composition;
(E) 0.2 to 45% weight, preferably 0.2 to 45% dry weight, fungal or bacterial spores, or fungus or bacteria liquid fermentation broth;
(F) 0.2 to 45% weight, preferably 0.2 to 45% dry % weight, fungi or bacteria spores, or fungi or bacteria liquid fermentation broth, or pesticides (herbicides, insecticides, nematicides or fungicides), or biostimulant compounds (terpenoids, proteins, lipopolysaccharides, polypeptides, polyamines or other signaling molecules), previously treated with 0.2 to 45% weight, preferably 0.8 to 45% weight, mineral or plant oil or a mixture;
(G) 0.1 to 10% weight, preferably 2%, of UV protector.

The method of preparing the above formulations comprises the following sequence of component add order: (i) addition of a microorganism in oil, (ii) addition of the inert agent, (iii) addition of emulsifiers, dispersants, moisturizing agents, and UVA/UVB protector; (iv) the optional addition of a second microorganism as fermentation broth in a final step of mixing to moisten the slurry obtained, or a pesticide (herbicides, insecticides, nematicides or fungicides) or powdered spores suspended or solubilized in water, oil (mineral or plant, and mixtures thereof) or organic solvents (alcohols, aliphatic, aromatic or halogenated hydrocarbons, alcohols, ketones or esters, and mixtures thereof) and previously treated with surfactants, the same mentioned above; (v) extrusion and drying of the slurry obtained in a fluidized bed.

If the addition of other components is needed, e.g., anti-foaming, these should be added after the final step for each formulation example described above.

Further according to the method, the resulting formulation comprises the addition of the components in the following ratio: from 1 to 30% (m:m) powdered microorganism suspended in oil; from 5 to 10% (m:m) emulsifiers; from 5 to 10% (m:m) dispersants; 5 to 10% (m:m) moisturizing agents; from 20 to 90% (m:m) inert agents; 1-2% UVA/UVB protector; from 1 to 10% (m:m) a second microorganism as a fermentation broth or suspended powder spores or pesticide (herbicides, insecticides, nematicides or fungicides) or biostimulant compounds (terpenoids, proteins, lipopolysaccharides, polypeptides, polyamines or other signaling molecules) soluble in water, oil (mineral or plant, and mixtures thereof) or organic solvents (alcohols, aliphatic, aromatic or halogenated hydrocarbons, alcohols, ketones or esters, and mixtures thereof).

The slurry obtained must be homogenized and there must be no addition of water.

The slurry should then be extruded into a 0.2 to 2 mm mesh, preferably 0.8 to 1 mm.

The extruded slurry must be dried in a fluidized bed for a period of time between 30 seconds and 60 minutes, preferably between 10 and 15 minutes, at a temperature between 25 and 100° C., preferably between 30 and 40° C.

The granules must be stored in a dry environment and at room temperature (up to 30° C.).

From this method, formulation samples were developed and showed better physicochemical performance in relation to the market reference brand. Example 2 describes the comparisons about the most difficult physicochemical parameter of improvement in water-dispersible granule-type formulations developed from the first method described in Example 1.

### Example 2 Suspensibility of Samples of Dispersible Granule-Type Formulations Containing Trichoderma harzianium Spores

| Sample | Microorganism concentration | Suspensibility |
|---|---|---|
| Market reference | 28% | 47% |
| Sample 1 | 30% | 60% |
| Sample 2 | 30% | 63% |
| Sample 3 | 30% | 78% |
| Sample 4 | 30% | 81% |

In this example, newly fermented Trichoderma harzianium spores from a solid formulation made available by the Instituto Mato-grossense do Algodão (IMA-MT) were used.

Comparisons were made with a market reference brand (28% dry mass Trichoderma asperellum).

The main difference between the samples refers to the biocompatible surfactants applied in the formulation to improve the physicochemical performance. As previously mentioned, some surfactant components can be biocompatible and, thus, can be applied in formulations containing microorganisms in order to improve physicochemical properties of microbiological actives.

### Example 3—Viability of Samples of Dispersible Granule-Type Formulations Containing Trichoderma harzianium Spores

| | Viability (% viable |
|---|---|
| Sample | conidia/total conidia) |
| Newly fermented spores | 58% |
| Sample 1 | 55% |
| Sample 2 | 50% |
| Sample 3 | 48% |
| Sample 4 | 47% |

The improvement in viability also results from the proper adjustment of the oil to be used in the encapsulation so that it shows a required HLB closer to the value also required by the microorganism. This will ensure better effectiveness in the encapsulation and, thus, improvement in shelf life and maintenance of the initial viability of the microorganism until field application. In this example, mineral oil, whose required HLB is 10, was used as a base, in contrast to the HLB required by dry spores of Trichoderma harzianium (−7.5).

It is noteworthy that shelf life and maintenance of viability can be optimized through the use of a mixture of mineral oil with other types of oil that reach the HLB required by the microorganism.

The microorganism concentration is determined by the ratio between microorganisms used and encapsulation oil. This ratio defines the amount of free formulation for the inclusion of inert agents and surfactants. Thus, the ratio between microorganisms and oil must total a maximum of 80% of the final constituents of the formulation in order to maintain space for inert agents and surfactants and, thus, not harming its physical-chemical parameters.

The formulations of the present invention can be applied to the soil, directly or through a gravity, sprinkler, drip, infiltration, center pivot irrigation, or submersion irrigation system. They can also be applied directly on the plants by means of spraying, or through seed treatment.

The formulations described herein show constant and consistent viability results within seven months of formulation. The maintenance of this viability is directly related to the efficiency of encapsulation in oil, that is, the greater the efficiency, the lower the loss of viability of the microorganisms throughout the shelf life.

1. A formulation of water-dispersible granules containing actives, comprising:
(i) 1 to 30% (m:m) of at least one microorganism suspended in oil, wherein the at least one microorganism is fungi or bacteria or viruses in an oil suspended powder or fermentation broth;
(ii) 20 to 30% (m:m) of oil emulsifier, wherein the oil emulsifier comprises mineral, plant oil or their mixtures;
5 to 10% (m:m) of
dispersants, wherein the dispersants comprise glycols, ethoxylated alcohols, ethoxylated phenols, ethoxylated polyols, ethoxylated and propoxylated polyols, sorbitan esters, sorbitan esters ethoxylates, ethoxylated and propoxylated sorbitan esters, and mixtures thereof,
(iv) 5 to 10% (m:m) of moisturizing, wherein the moisturizing comprises acetates, ethoxylated alcohols and block copolymers,
(v) 1-2% UVA/UVB protector,
(vi) 20 to q.s.p (m:m) of inert agents, wherein the inert agents make up polysaccharides, such as chitosan, cellulose, starch, or minerals
may also comprise (vii) other microorganisms and/or pesticides, or biostimulant compounds, suspended or solubilized in water, oil or organic solvents and previously treated with surfactants.

2. (canceled)

3. (canceled)

4. (canceled)

5. (canceled)

6. (canceled)

7. (canceled)

8. The formulation according to claim 1, characterized in that other microorganisms comprise bacteria, fungi or viruses.

9. The formulation according to claim 1, characterized in that the pesticide comprises insecticides.

10. The formulation according to claim 1, characterized in that the pesticide comprises fungicides.

11. The formulation according to claim 1, characterized in that the pesticide comprises nematicides.

12. The formulation according to claim 1, characterized in that it is applied to the soil, directly or through a gravity irrigation, sprinkler, drip, infiltration, central irrigation pivot, or submersion system.

13. The formulation according to claim 1, characterized in that it is applied directly on the plants by means of sprinkling.

14. The formulation according to claim 1, characterized in that it is applied through seed treatment.

15. A method of preparing formulation of water dispersible granules containing microbiological actives according to claim 1, characterized in that it comprises the following sequence of component add order:
(i) add an oil microorganism, (ii) add the inert agent, (iii)
add the emulsifiers, dispersants, moisturizing, and UVA/UVB protector; (iv) the optional add a second microorganism as a fermentation broth in a final stage of mixing to moisten the obtained slurry, or of a pesticide (herbicides, insecticides, nematicides or fungicides) or powdered spores suspended or
solubilized in water, oil or organic solvents and previously treated with surfactants; (v) extrusion and drying of the slurry obtained in a fluidized bed.

16. The method according to claim 15, characterized in that the resulting formulation comprises the addition of the components in the following ratio: 1 to 30% (m:m) powdered oil-suspended microorganism; 5 to 10% (m:m) emulsifiers; 5 to 10% (m:m) dispersants; 5 to 10% (m:m) moistening; 20 to 90% (m:m) inert agent; 1-2% UVA/UVB protector; 1-10% (m:m) a second microorganism as a fermentation broth or oil-, water-suspended powdered spores or water-soluble pesticide, or organic solvents.

17. The method according to claim 15, characterized in that the slurry extrusion needs a mesh of 0.2 to 2 mm diameter.

18. The method according to claim 15, characterized in that the drying of extruded slurry in a fluidized bed is performed in a time period between 30 seconds and 60 minutes, at a temperature between 25 and 100° C.
